(19) United States
(12) Patent Application Publication
(10) Pub. No.: US 2024/0041943 A1
(43) Pub. Date: Feb. 8, 2024

(54) METHOD OF ISOLATION, PURIFICATION, AND CHARACTERIZATION OF HEPARIN-LIKE SUBSTANCES FROM SNAIL MUCUS (ACHATINA FULICA) AND ITS USES

(71) Applicant: ADEN INTERNATIONAL CO.,LTD., Bangkok (TH)

(72) Inventor: Prachya KONGTAWELERT, Chiang Mai (TH)

(73) Assignee: ADEN INTERNATIONAL CO.,LTD., Bangkok (TH)

(21) Appl. No.: 18/265,655

(22) PCT Filed: Jan. 8, 2021
(86) PCT No.: PCT/TH2021/000001

§ 371 Date: Jun. 6, 2023

(51) Int. Cl. A61K 35/618; A61P 35/00

(52) U.S. Cl. CPC A61K 35/618; A61P 35/00

(57) ABSTRACT

Heparin-like substances (HLS) have their structure similar with heparin, the highly sulfated glycosaminoglycan (GAGs). The polysaccharide, a product of mast cells, is isolated from animal tissues (pig intestinal mucosa, bovine lung), and owes its anticoagulant activity, activates antithrombin and proteases inhibitor. Disclosed herein are novel methods of isolation, purification and characterization of HLS from snail mucus (Achatina fulica), and its uses. This HLS can be used to develop a simple, sensitive and specific assay for its biological activities of anti-SARs-CoV-2 (COVID-19) and anti-PD-L1 on the breast cancer cell.

### TECHNICAL FIELD

Pharmaceutical

Biological

### BACKGROUND ART

Heparin-like substances (HLS) are closely related Glycosaminoglycans (GAGs) consisting of alternating uronic acid (IdoA or beta-D-glucuronic acid (GlcA) (1→4) glycosidically linked to glucosamine (alpha-D-N-Sulfoglucosamine (GlcNS) or alpha-D-N-Sulfoglucosamine (GlcNAc) disaccharides repeating unit (Linhardt, 2003). HLS have been described in various clinical situations, they are generally attributed to circulating glycosaminoglycans, primarily heparin (HP) and dermatan sulfate (DS). The highly sulfated glycosaminoglycans, heparin, has been used as and antithrombotic for 102 years old drug, and still remains one of the most widely prescribed drugs today. As a medication it is used as anticoagulant (blood thinner). Specifically it is also used in the treatment of heart attacks and unstable angina, it is given by injection into a vein or under the skin. Other uses include inside test tubes and kidney dialysis machines.

Heparin, also known as unfractionated heparin (UFH), is produced by basophils and mast cells in all mammals. Commercial preparations are now most common derived from mucosal intima of pig (porcine) intestine with long production cycle however several countries, including Argentina, Brazil and India still allow bovine-derived heparin for religious reasons. In the past still had not yet ever studied the novel method of isolation, purification and characterization of heparin-like substances from snail mucus (Achatina fulica) and its uses, same as this said inventions. Due to the techniques are poorly described in records.

The invention provides a kind of simple to operate with short production cycle under novel method of isolation, purification, and characterization of HLS from snail mucus (Achatina fulica) as new alternative source which is free of any religious, health reason concerns and risk protection for future heparin & HLS shortages. An alternative resource for the raw material, which is able to be farmed in the house-hold and organic environment. This resource can be managed as industrial and agricultural harvesting subjected to additional uses and easily to isolate and purity the active fraction from the starting raw material. This invention has showing relative testing report of HLS use for anti-SARS-CoV-2 and anti-PD-L1 activity on the breast cancer cell including the anti-migrating activity of the breast cancer cell.

J Y van der Meer (2017). From Farm to Pharma: An Overview of Industrial Heparin Manufacturing Methods, this paper has indicated many chemicals were added in porcine/bovine/ovine heparin manufacturing process closely related to health reason concerns.

Haiying Liu et al. (2009). Lessons learned from the contamination of heparin, the paper highlight the heparin contamination crisis of heparinoids as potential heparin contaminants.

Szajek A. Y. et al. (2016) The US regulatory and pharmacopeia response to the global heparin contamination crisis, the paper reported porcine heparin manufacturing processes have not changed substantially since its introduction.

PAT-CN105, 001, 353A disclose refining optimization technology for crude heparin sodium without any crude heparin isolation method which is longer and more complicated process.

PAT-EP0113040A2 disclose an ultrafiltration process with two membranes for the purification and fractionation of heparin without any descripted the process is better than prior art.

PAT-KR0170064B1 disclose snail extracts containing acharan sulfate, the invention use snail tissues source with different extraction method. GAG precipitation by Trichloroacetic acid (TCA), Potassium acetate, Cetylpyridinium chloride may be found hazardous residues in the process.

PAT-IN2502DEL1996A disclose a process for the isolation of a new highly specific 9-O-acetllated sialoglycoconjugate binding lectin (Achatinin-H) from Achatina fulica snail useful for diagnosis of visceral leishmaniasis, use haemolymph source with different isolation method.

U Lindahl (2020). Heparin—An old drug with multiple potential targets in Covid-19 therapy, the reports show that treatment with low-molecular weight heparin (LMWH) decreases mortality in critically ill patients.

Andrade et al. (2013). A heparin-like compound isolated from a marine crab rich in glycuronic acid 2-O-sulfate present low anticoagulant activity.

Li Fu et al. (2016). Bioengineered heparins and heparan sulfates disclose the use of recombinant technology in the chemoenzymatic synthesis and metabolic engineering to solve their inherent impurities, the limited of source tissues, the poor control of manufacturing processes.

Griffin et al. (1995). Isolation and characterization of Heparan sulfate from crude porcine intestinal mucosal peptidoglycan heparin, disclose is longer and more complicated process.

Linhardt et al. (1995). Dermatan sulfate as a potential therapeutic agent of anticoagulant and antithrombotic agents.

### DISCLOSURE OF INVENTION

This invention is an innovative strategic research to discovery the alternative source of heparin-like substances (HLS) from snail mucus, Achatina fulica, and its new biological activities as anti-PD-L1, and anti-SARS-CoV2 (COVID-19) infection, the invention provides a kind of simple and easily to isolate, purify & characterize the active fraction from the starting raw material with short production cycle, product yield is high, reliable in quality which is free of any religious, health reason concerns, risk protection for future heparin & HLS shortages and the most important advantage of this invention is the source of raw material can be used to produce unlimited amount of the drug.

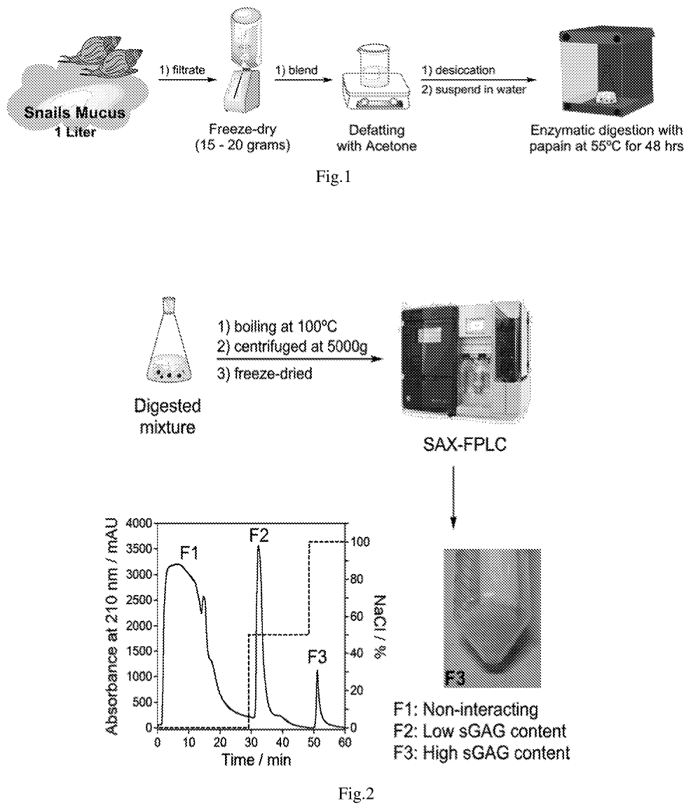

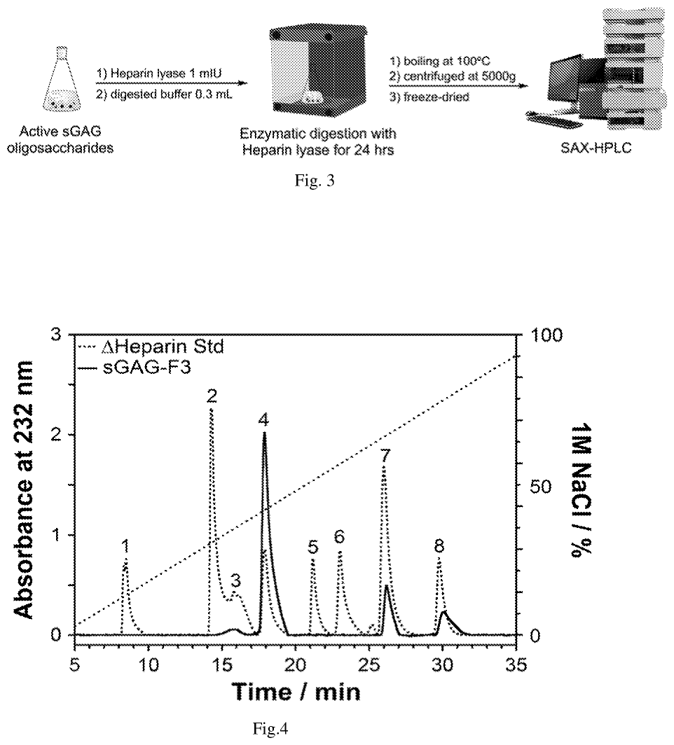

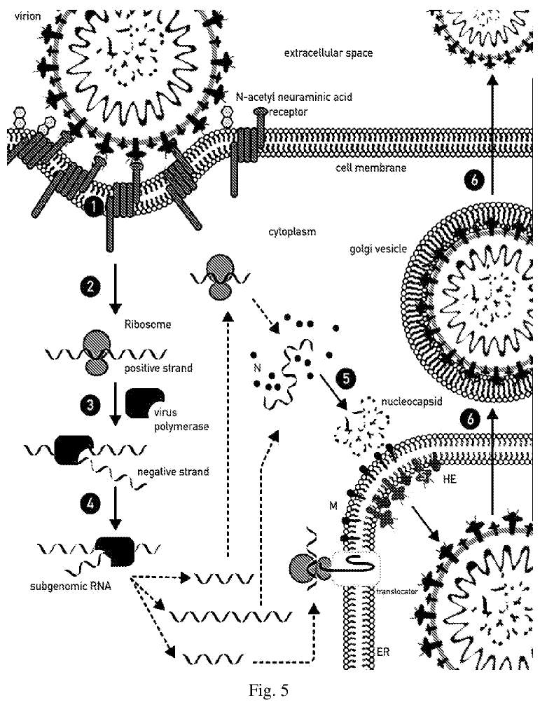

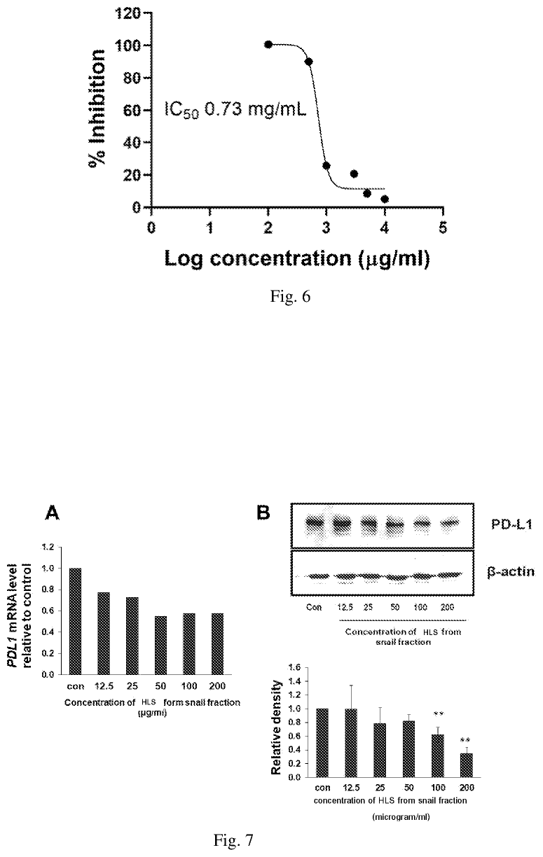

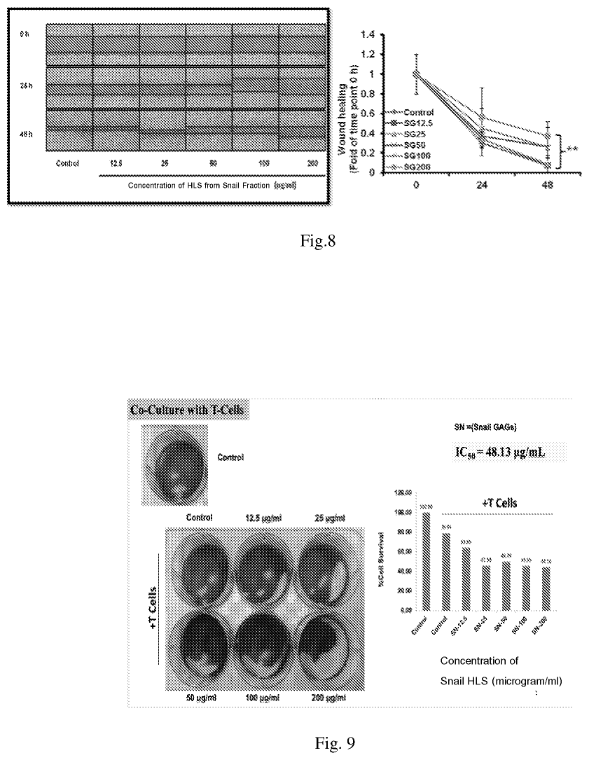

For achieving the above object, technical scheme of the present invention are:
(1) We have developed a novel method to isolate, purity and characterize heparin-like substances (HLS) from snail mucus (Achatina fulica) as show in FIGS. 1 and 2 as following.
1.1 Preparation of sulfated glycosaminoglycans (GAGs) from snail mucus
1 Liter of Mucus was collected from the foot & mantle of live snails, then freeze-dried by lyophilization process. The dried mucus powders were defatted by 3-volumes of acetone with shaking overnight. After freeze drying, the mucus powders (15˜20 grams) were suspended with 5-volumes of sodium acetate buffer (pH 5.5) containing 5 mM EDTA and 5 mM cysteine. 50 mg (3 times each) of papain enzyme (3.2 units/mg solids) was added. Then, the reaction mixture was incubated at 55° C. for 48 hours with shaking. The digestion mixture was stopped by heating at 100° C. for 5 min, then the suspension was centrifuged at 5000 g for 30 min at room temperature and the supernatant, which contained the GAGs, was collected.
1.2 Isolation and purification of sulfated glycosaminoglycans (GAGs) from digested snail mucus the papain digested samples were isolated and purified by an anion-exchange chromatography on DEAE cross-linked beaded agarose column (HiTrap DEAE FF, GE healthcare). The elution was performed in a stepwise concentration in range of 0.1 M-1.0 M NaCl in 50 mM sodium acetate buffer (pH 5.5). The elution was monitored at 210 nm and the flow rate was set at 0.5 mL/min. Three eluent fractions were collected, (1) non-interacting fraction (F1, 1.2% w/w yield), (2) low sulfated GAG content (F2, 6.9% w/w yield), and (3) high sulfated GAG content (F3, 14% w/w yield). The isolated fraction was exhaustively desalted by chromatography on HiTrap desalting column (GE healthcare) and freeze-dried to give a pale-yellow color powder.
1.3 The characterization of repeating disaccharides has been discovered.
The specific disaccharides composition of the isolated GAG from snail mucus can be investigated by using enzymatic digestion and HPLC. The active sulfated GAG fraction (F3) was performed as shown in FIG. 3 as following procedures. The high sulfated GAG content of F3 oligosaccharides was depolymerized using 1 mIU of heparin lyase II (heparintinase II) and III (heparintinase I) in 0.3 mL of 50 mM Tris-HCl buffer (pH 7.2) and 10 mM CaCl₂ for 24 hours at 37° C. with shaking. After which the reaction mixture was heated in a boiling water for 5 minutes, centrifuged at 5000 g for 10 min, and freeze-dried. The heparin lyase digested products were injected on an analytical SAX-HPLC column (Phenomenex, 0.46×25 cm, Torrells CA) to monitor the reaction. A linear gradient of 0.1-1.0 M NaCl was performed over a 40 min period at a flow rate of 1.0 mL/min and the detection was set at 232 nm for monitoring the unsaturated disaccharides of uronic acid. As per FIG. 4, the major peak of active sulfated GAG fraction (F3) disaccharide observed at peak 4 (17.89 min) with over 73% composition which corresponds to the repeating disaccharide unit of acharan sulfate, ΔHexA(2S)-GlcNAc. In additions, ΔHexA(2S)-GlcNAc(6S) (peak 7) and ΔHexA(2S)-GlcNSO3(6S) (peak 8) were also found with 15% and 12% components, respectively. All is called “Snail heparin-like substances (Snail HLS)” hereinafter
(2) The in vitro investigation of anti-SARS-CoV-2 was reported.
The life-cycle of the SARS-CoV-2 has been reported, and each step of virus infection and replication has been targeted for the drug discovery. Especially, the first step as virus particles use the Spike protein binding to the receptor which is ACEII (angiotensin converting enzyme II) as shown in FIG. 5 Antiviral activity was evaluated on the basis of the inhibition of Spike-ACE2 binding by competitive immunoassay. The inhibitory activity with mean 50% inhibitory concentration (IC₅₀) was calculated (FIG. 6). The data indicate that Snail HLS showed the potent inhibitory activity against SAR-CoV-2 spike RBD region.
(3) The anti-PD-L1 activity on the breast cancer cell line (MDA-MB231) was reported. Inhibition of Programmed cell death ligand 1 (PD-L1) and programmed cell death 1 (PD-1) immune checkpoints by monoclonal antibodies has shown success in cancer. Binding of PD-L1 to PD-L1 inhibits T cells effector function, resulting in an immunosuppressive state. Expression of PD-L1 in cancer cells plays an important role in cancer immune escape and cancer progression. Development of compound downregulated PD-L1 expression was investigated. It was found that Snail HLS can exhibit the down-regulation of PD-L1 both gene expression and protein level in breast cancer cells, MDA-MB231 as shown in FIG. 7.
(4) The anti-migrating activity of the breast cancer cell was reported
The metastasis is the most characteristic stage of the cancer leading the dysfunction of the tissues and other organs throughout the body. We have investigated the inhibition of the cancer cell migration using the scrap-assay to investigate the properties of Snail HLS As shown in FIG. 8, it was found that Snail HLS from Achatina fulica was able to inhibit migration of the breast cancer cells using migration assay.
(5) Snail HLS increase ability the cytotoxicity effect of T-cell on MDA-MB231 cells was reported. T cells cytotoxicity to tumor cells assay. Peripheral blood mononuclear cells (PBMCs) derived from healthy donor screening will be collected from Blood Bank Section Maharaj Nakorn Chiang Mai Hospital, and PBMCs were isolated by differential density gradient centrifugation (Ficoll). To stimulate PBMC, 6 well plates were coated with anti-CD3 by pre-incubation of the plates with 1 μg/ml antibody in PBS for 4 h. Furthermore, were supplemented with soluble anti-CD28 antibody (1 μg/ml) and IL-2 (10 ng/ml) at the start of the culture experiment. After pretreatment of MDA-MB231 with or without Snail HLS (0-200 μg/ml) for 48 h, the medium was replaced and cancer cells were co-cultured with T-cell activation (The ratio of tumor cells to lymphocytes is 1:10). Activated T cells well was with PBS twice to remove T cells and then the living cancer cells were fixed and stained with crystal violet was dissolved with 20% acetic acid and absorbance was measured at 590 nm. To determine whether the down regulation of PD-L1 by Snail HLS on MDA-MB231 cells alters immune-mediated cytotoxicity, T cells isolated from healthy volunteer's peripheral blood mononuclear were incubated with MDA-MB231 cells pretreatment with Snail HLS for 48 h. After that co-culture for 24 h, the survived cancer cells were marked with crystal violet solution. Although T cells slightly decrease the survival of cancer cells in absence of Snail HLS compared with control (without T-Cells). The treatment of Snail HLS (200 μg/ml) reduced the survival of cancer cells approximately 33.78% in MDA-MB231 cells co-cultured with T-cells compared with control group (with T-cells) as showing in FIG. 9. The result findings suggested that T-cells was activated by cancer cells PD-L1 down regulation induced by Snail HLS and increased the killing effect on cancer cells.

### BRIEF DESCRIPTION OF DIAGRAM

FIG. 1. The diagram showing the method for the preparation of sulfated glycosaminoglycans from snail mucus.

FIG. 2. The method for the isolation and purification of sulfated glycosaminoglycans from digested snail mucus by ion-exchange chromatography.

FIG. 3. The method for the disaccharide composition analysis of active sulfated oligosaccharides by HPLC-UV detection.

FIG. 4. The HPLC chromatogram of the repeating disaccharides containing in the active sulfated GAGs fraction as called Snail HLS. The standard disaccharide (black dashed line) structures are indicated by number labels; 1: ΔHexA-GlcNAc, 2: ΔhexA-GlcNSO3, 3: ΔhexA-GlcNAc(6S), 4: ΔhexA(2S)-GlcNAc, 5: ΔhexA-GlcNSO3(6S), 6: ΔhexA(2S)-GlcNSO3, 7: ΔhexA(2S)-GlcNAc(6S), 8: ΔhexA(2S)-GlcNSO3(6S). Abbreviations: GlcNAc, N-acetylglucosamine; GlcNSO3, N-sulfated glucosamine; 2S and 6S are 2-O- and 6-O-sulfate groups, respectively; ΔhexA, unsaturated hexuronate residue formed at non-reducing end of disaccharides and oligosaccharides by eliminative lyase scission.

FIG. 5. The life-cycle of the SARS-CoV-2, and steps of the proliferation.

FIG. 6. The inhibition curve of Snail HLS on the binding of Spike protein. Data are fitted with sigmoidal model and the value of IC₅₀ was indicated.

FIG. 7. The effect of Snail HLS on the expression of PD-L1 in breast cancer cells, MDA-MB231 using the real-time PCR (A) and Western Blotting (B) (p<0.01).

FIG. 8. The effect of Snail HLS on the migration of the MDA-MB231 cells (p<0.01).

FIG. 9. The treatment of Snail HLS (200 μg/ml) reduced the survival of cancer cells approximately 33.78% in MDA-MB231 cells co-cultured with T-cells compared with control group (with T-cells).

1. A novel method to isolate, purify and characterize heparin-like substances (HLS) from snail mucus (Achatina fulica)
(1) Preparation of sulfated glycosaminoglycans (GAGs) from snail mucus: 1 Liter of Mucus was collected from the foot & mantle of live snails, then freeze-dried by lyophilization process. The dried mucus powders were defatted by 3-volumes of acetone with shaking overnight. After freeze drying, the mucus powders (15˜20 grams) were suspended with 5-volumes of sodium acetate buffer (pH 5.5) containing 5 mM EDTA and 5 mM cysteine. 50 mg (3 times each) of papain enzyme (3.2 units/mg solids) was added. Then, the reaction mixture was incubated at 55° C. for 48 hours with shaking. The digestion mixture was stopped by heating at 100° C. for 5 min, then the suspension was centrifuged at 5000 g for 30 min at room temperature and the supernatant, which contained the GAGs, was collected.
(2) Isolation and purification of sulfated glycosaminoglycans (GAGs) from digested snail mucus: the papain digested samples were isolated and purified by an anion-exchange chromatography on DEAE cross-linked beaded agarose column (HiTrap DEAE FF, GE healthcare). The elution was performed in a stepwise concentration in range of 0.1 M-1.0 M NaCl in 50 mM sodium acetate buffer (pH 5.5). The elution was monitored at 210 nm and the flow rate was set at 0.5 mL/min. Three eluent fractions were collected, (1) non-interacting fraction (F1, 1.2% w/w yield), (2) low sulfated GAG content (F2, 6.9% w/w yield), and (3) high sulfated GAG content (F3, 14% w/w yield). The isolated fraction was exhaustively desalted by chromatography on HiTrap desalting column (GE healthcare) and freeze-dried to give a pale-yellow color powder.
(3) The characterization of repeating disaccharides has been discovered: the specific disaccharides composition of the isolated GAG from snail mucus can be investigated by using enzymatic digestion and HPLC. The active sulfated GAG fraction (F3) was performed. The high sulfated GAG content of F3 oligosaccharides was depolymerized using 1 mIU of heparin lyase II (heparintinase II) and III (heparintinase I) in 0.3 mL of 50 mM Tris-HCl buffer (pH 7.2) and 10 mM CaCl₂ for 24 hours at 37° C. with shaking. After which the reaction mixture was heated in a boiling water for 5 minutes, centrifuged at 5000 g for 10 min, and freeze-dried. The heparin lyase digested products were injected on an analytical SAX-HPLC column (Phenomenex, 0.46×25 cm, Torrells CA) to monitor the reaction. A linear gradient of 0.1-1.0 M NaCl was performed over a 40 min period at a flow rate of 1.0 mL/min and the detection was set at 232 nm for monitoring the unsaturated disaccharides of uronic acid. The major peak of active sulfated GAG fraction (F3) disaccharide observed at peak 4 (17.89 min) with over 73% composition which corresponds to the repeating disaccharide unit of acharan sulfate, ΔHexA(2S)-GlcNAc. In additions, ΔHexA(2S)-GlcNAc(6S) (peak 7) and ΔHexA(2S)-GlcNSO3(6S) (peak 8) were also found with 15% and 12% components, respectively. All is called “Snail heparin-like substances (Snail HLS)” hereinafter.

2. Snail HLS use for in vitro investigation of anti-SARS-CoV-2 was reported. Antiviral activity was evaluated on the basis of the inhibition of Spike-ACE2 binding by competitive immunoassay. The inhibitory activity with mean 50% inhibitory concentration (IC₅₀) was calculated. The data indicated that Snail HLS showed the potent inhibitory activity against SAR-CoV-2 spike RBD region.

3. Snail HIS use for anti-PD-L1 activity on the breast cancer cell line (MDA-MB231) was reported. Inhibition of Programmed cell death ligand 1 (PD-L1) and programmed cell death 1 (PD-1) immune checkpoints by monoclonal antibodies has shown success in cancer. Binding of PD-L1 to PD-L1 inhibits T cells effector function, resulting in an immunosuppressive state. Expression of PD-L1 in cancer cells plays an important role in cancer immune escape and cancer progression. Development of compound downregulated PD-L1 expression was investigated. It was found that Snail HLS can exhibit the down-regulation of PD-L1 both gene expression and protein level in breast cancer cells, MDA-MB231.

4. Snail HLS use for anti-migrating activity of the breast cancer cell was reported, we have investigated the inhibition of the cancer cell migration using the scrap-assay to investigate the properties of Snail HLS, it was found that Snail HLS from Achatina fulica was able to inhibit migration of the breast cancer cells using migration assay.

5. Snail HIS use for increase ability the cytotoxicity effect of T-cell on MDA-MB231 cells was reported. T cells cytotoxicity to tumor cells assay, peripheral blood mononuclear cells (PBMCs) derived from healthy donor screening will be collected from Blood Bank Section Maharaj Nakorn Chiang Mai Hospital, and PBMCs were isolated by differential density gradient centrifugation (Ficoll). To stimulate PBMC, 6 well plates were coated with anti-CD3 by pre-incubation of the plates with 1 μg/ml antibody in PBS for 4 h. Furthermore, were supplemented with soluble anti-CD28 antibody (1 μg/ml) and IL-2 (10 ng/ml) at the start of the culture experiment. After pretreatment of MDA-MB231 with or without Snail HIS (0-200 μg/ml) for 48 h, the medium was replaced and cancer cells were co-cultured with T-cell activation (The ratio of tumor cells to lymphocytes is 1:10). Activated T cells well was with PBS twice to remove T cells and then the living cancer cells were fixed and stained with crystal violet was dissolved with 20% acetic acid and absorbance was measured at 590 nm. To determine whether the down regulation of PD-L1 by Snail HLS on MDA-MB231 cells alters immune-mediated cytotoxicity, T cells isolated from healthy volunteer's peripheral blood mononuclear were incubated with MDA-MB231 cells pretreatment with Snail HLS for 48 h. After that co-culture for 24 h, the survived cancer cells were marked with crystal violet solution. Although T cells slightly decrease the survival of cancer cells in absence of Snail HLS compared with control (without T-Cells). The treatment of Snail HLS (200 μg/ml) reduced the survival of cancer cells approximately 33.78% in MDA-MB231 cells co-cultured with T-cells compared with control group (with T-cells). The result findings suggested that T-cells was activated by cancer cells PD-L1 down regulation induced by Snail HLS and increased the killing effect on cancer cells.
